(19) United States
(12) Patent Application Publication
(10) Pub. No.: US 2018/0311016 A1
(43) Pub. Date: Nov. 1, 2018

(54) Device Using Regeneration Membrane for Dentistry

(71) Applicants: Ming-Yuan Shao, Taipei (TW); Chih-Hsien Lin, Taipei (TW)

(72) Inventors: Ming-Yuan Shao, Taipei (TW); Chih-Hsien Lin, Taipei (TW)

(21) Appl. No.: 15/499,998

(22) Filed: Apr. 28, 2017

(51) Int. Cl. A61C 8/02; A61C 13/15; A61N 5/06; A61F 2/46; A61F 2/28; A61L 27/24

(52) U.S. Cl. CPC A61C 8/0006; A61C 19/003; A61N 5/062; A61N 2005/0661; A61F 2/2846; A61L 27/24; A61N 2005/0606; A61F 2/4601

(57) ABSTRACT

A regeneration membrane is provided for dentistry. The traditional thin collagen is turned into a flowable and shapeable collagen gel. A photo-crosslinking agent (i.e. riboflavin (vitamin B2)) is added in a biomedical-level collagen gel to obtain the shapeable collagen gel. The shapeable collagen gel is filled in a pre-filled syringe. The shapeable collagen gel is squeezed to a destined position for paving and shaping. Then, crosslinking is processed through UV illumination for curing. Flowability is achieved for applying. After applying, solidification is finished through illumination to turn liquid into solid. Thus, a regeneration membrane is formed with biological tolerance.

### TECHNICAL FIELD OF THE INVENTION

The present invention relates to a regeneration membrane; more particularly, relates to turning the traditional thin collagen membrane into a flowable collagen gel, where flowability is achieved for applying and, after applying, crosslinking is processed with illumination for curing to turn liquid into semisolid to form a regeneration membrane with biological tolerance.

### DESCRIPTION OF THE RELATED ARTS

Guided bone regeneration (GBR) surgery is commonly used in dentistry to guide regeneration of atrophy tissue like alveolar bone for recovery of periodontal structures and dental treatment follow-ups such as implant. During the GBR surgery, for isolating the gingival epithelial tissue from invading the defect area of alveolar bone, the current commonly used practice is to place a regeneration membrane (or artificial periosteum) between the epithelia and alveolar bone to gain time for recovery. At the same time, artificial bone graft is filled into the defect area of alveolar bone to help the regeneration of alveolar bone.

Conventional regeneration membranes can be divided into non-absorbable ones and absorbable ones. Non-absorbable regeneration membranes are usually made of Gore-TEX®, Millipore® and Biobarrier®, where special biochemical materials, like e-polytetrafluoroethylene (e-PTFE), may be contained to be of significant help to bone integration for dental implant. Not only the invasion of epithelial cells and fibroblasts into the defect area is inhibited; but also the growth of bone cells is enhanced. Other benefits include cell occlusivity, space making, tissue integration, biocompatibility, good stability and reliability. However, its disadvantage is that the GBR surgery using the regeneration membrane would likely expose the surgical wound and cause wound infection, which causes bad surgery effect. If subsequent implant is required, a second surgery is not avoidable for removing the nonabsorbable regeneration membrane.

According to different material compositions, the absorbable regeneration membranes can be divided into collagen regeneration membranes, polymer regeneration membranes, and regeneration membranes of other materials. At present, collagen is the main material for regeneration membranes. The advantage is that such collagen material is placed in human body and can be gradually absorbed by the body over time. It does not need a second surgery to remove the regeneration membrane. It has good blood permeability for maintaining the blood circulations between the tissues. Yet, its disadvantage is that such regeneration membrane does not have sufficient supporting force and is hard to be solidified. This kind of regeneration membrane is basically a flaky one horizontally placed between the epithelia and the alveolar bone. It is hard for shaping so that it is difficult to maintain the growth space of the alveolar bone. The alveolar bone may be absorbed by human body before being fully grown and, therefore, the desired effect is not achieved.

The polymer regeneration membranes are mainly made by polymerization with polylactic acid (PLA) and polyglycolic acid. These materials can be completely absorbed and non-toxic. But polylactic acid is strongly rigid with poor toughness, whose application becomes limited. Because its degradation is too fast, its use life is short. Moreover, its acidic degradation products would cause decrease in the local pH value, which may easily lead to aseptic inflammation in patients and further lead to bad effect on tissue healing.

The currently developed biodegradable and highly-biocompatible materials are usually fast decomposed in human body. Quite often, they do not stay long enough in human body to meet the time required for recovery of the tissues.

Hence, the prior arts do not fulfill all users' requests on actual use.

### SUMMARY OF THE INVENTION

The main purpose of the present invention is to turn the traditional thin collagen membrane into a flowable collagen gel, where a photo-crosslinking agent (i.e. riboflavin (vitamin B2)) is added in a biomedical grade collagen gel to obtain a shapeable collagen gel to be filled in a pre-filled syringe (further added with a needle or not depending on demand); the shapeable collagen gel is squeezed to a destined position for paving and shaping; at last, crosslinking is processed through UV illumination for curing with flowability achieved for applying; and, after applying, solidification is finished through illumination to turn liquid into semisolid for forming a regeneration membrane with biological tolerance.

To achieve the above purpose, the present invention is a device using a regeneration membrane for dentistry, comprising a container and a shapeable collagen gel, where the container has a containing space; the shapeable collagen gel is obtained by mixing a photo-crosslinking agent and a collagen gel; the photo-crosslinking agent and the collagen gel are crosslinked and solidified by being illuminated with an ultraviolet (UV); the shapeable collagen gel is contained in the containing space to be sent to a surgical site; and the shapeable collagen gel isolates the surgical site from adjacent gingival connective tissues and epithelia to guide tissue regeneration with protection. Accordingly, a novel device using a regeneration membrane for dentistry is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the structural view showing the preferred embodiment according to the present invention; and

FIG. 2 is the view showing the state-of-use of the present invention.

### DESCRIPTION OF THE PREFERRED EMBODIMENT

The following description of the preferred embodiment is provided to understand the features and the structures of the present invention.

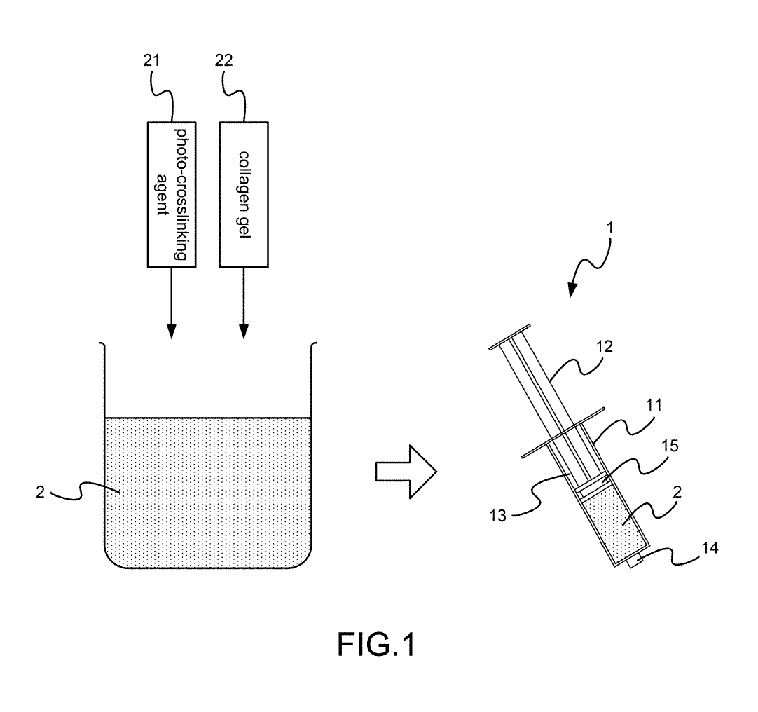

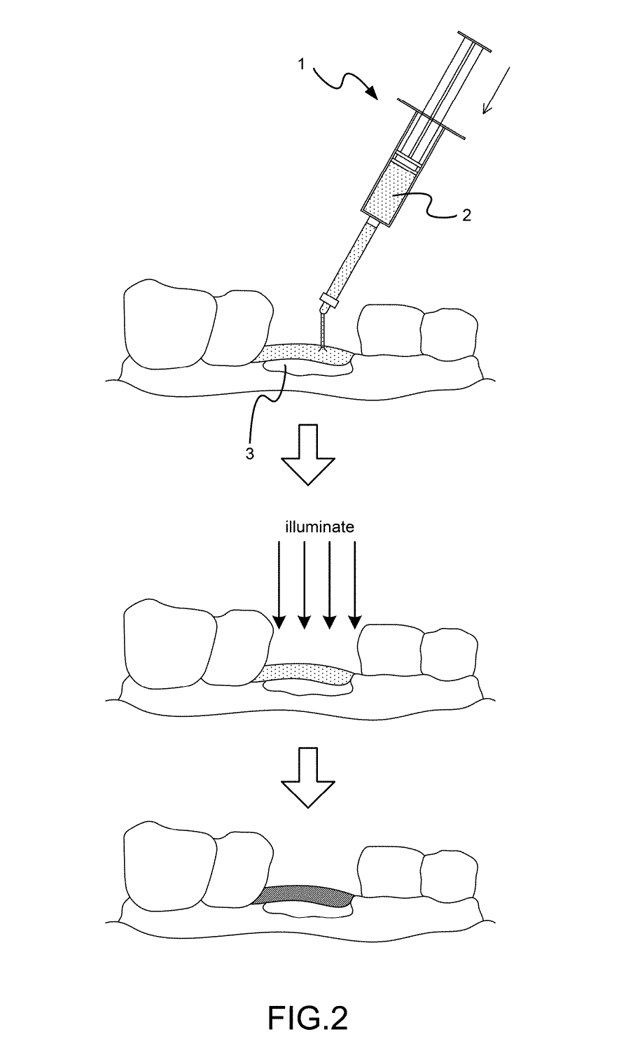

Please refer to FIG. 1 and FIG. 2, which are a structural view showing a preferred embodiment according to the present invention; and a view showing a state-of-use of the present invention. As shown in the figures, the present invention is a device using a regeneration membrane for dentistry, comprising a syringe 1 and a shapeable collagen gel 2.

The syringe 1 comprises a syringe body 11 and a push bar 12. The syringe body 11 has a containing space 13 with two ends communicating exteriorly. An opening at an end of the syringe body 11 is extended and gradually shrunk to form a connecting part 14. The connecting part 14 further has a needle holder with a needle (not shown in the figures). An end of the push bar 12 has a pushing part 15. The push bar 12 is inserted into the containing space 13 of the syringe body 11 at an end by the pushing part 15 and moves relatively with the syringe body 11.

The shapeable collagen gel 2 is contained between the containing space 13 and the pushing part 15 of the syringe 1. The shapeable collagen gel 2 is formed by mixing a photo-crosslinking agent 21 and a collagen gel 22. Based on a customer-specific requirement, the shapeable collagen gel 2 has liquidity adjusted in advance by UV illumination. The photo-crosslinking agent 21 is riboflavin (vitamin B2).

Thus, a novel device using a regeneration membrane for dentistry is obtained.

Before applying the present invention, the shapeable collagen gel 2 formed by mixing the photo-crosslinking agent 21 and the collagen gel 22 are obtained. The shapeable collagen gel 2 has a concentration of 3 percents (%) and needs 3 minutes (min) for crosslinking and solidifying. Hence, the shapeable collagen gel 2 is pretreated with UVA illumination for 2.5 min to obtain a crosslinking rate of 80%. Then, the shapeable collagen gel 2 is loaded in the syringe 1. The pressing part 15 is rubber-based to liquid-tightly combine the containing space 13 of the syringe body 11 and the push bar 12. On applying the present invention, the shapeable collagen gel 2 is injected into a surgical site 3 by using the syringe 1. The shapeable collagen gel 2 isolates the surgical site 3 from adjacent gingival connective tissues and epithelia to guide tissue regeneration with protection.

After being pretreated (illuminated), the shapeable collagen gel 2 becomes denser for facilitating more easily, so that the physician may use less time for illumination. Because the shapeable collagen gel 2 is pretreated with a crosslinking rate of 80% achieved, the physician simply needs to finish the remaining 20% crosslinking job with time significantly reduced.

Therein, since the collagen gel has already contained the crosslinking agent (i.e. riboflavin (vitamin B2)) within, crosslinking happens rapidly after UV illumination and produce the following phenomena:

1. Curing; and

2. Increasing the crosslinking rate of collagen. The crosslinking makes the collagen fight against enzyme for erosion and decomposition, and the survival time in vivo (i.e. biological tolerance) is increased.

From the above, it is known that the present invention turns the traditional thin collagen into a flowable collagen gel. The key point is to apply riboflavin (vitamin B2) in the biomedical-level collagen gel for forming the shapeable collagen gel to be filled in a pre-filled syringe (further added with a needle or not depending on demand). Then, the shapeable collagen gel is squeezed to a destined position for paving and shaping. At last, crosslinking is processed through UV illumination for curing with flowability achieved for applying. After applying, solidification is finished through illumination to turn liquid into solid for forming a regeneration membrane with biological tolerance.

To sum up, the present invention is a device using a regeneration membrane for dentistry, where riboflavin (vitamin B2) is applied in a biomedical-level collagen gel for forming the shapeable collagen gel to be filled in a pre-filled syringe; then, the shapeable collagen gel is squeezed to a destined position for paving and shaping; at last, crosslinking is processed through UV illumination for curing with flowability achieved for applying; and, after applying, solidification is finished through illumination to turn liquid into solid for forming a regeneration membrane with biological tolerance.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

1. A device using a regeneration membrane for dentistry, comprising
a container having a containing space and
a shapeable collagen gel mixture comprising a mixture of a photo-crosslinking agent and a 2-80% concentration collagen gel, wherein the shapeable collagen gel mixture is 60-80% crosslinked and solidified by pre-treatment illumination with ultraviolet (UV) and wherein said pre-treated shapeable collagen gel mixture is then loaded in said containing space and said pre-treated shapeable collagen gel mixture isolates a surgical site from adjacent gingival connective tissues and epithelia to guide tissue regeneration with protection.

2. The device according to claim 1, wherein said container is a syringe, comprising a syringe body and a push bar; said syringe body has said containing space with two ends communicating exteriorly; and an end of said push bar has a pushing part; said push bar is inserted into said containing space of said syringe body by said pushing part at an end and moves relatively with said syringe body; and said pre-treated shapeable collagen gel mixture is contained between said containing space and said pushing part of said syringe.

3. The device according to claim 2, wherein an opening at an end of said syringe body defines a connecting part.

4. The device according to claim 3, wherein said connecting part has a needle holder with a needle.

5. The device according to claim 2, wherein said pushing part is rubber-based to liquid-tightly combine said containing space of said container and said push bar.

6. The device according to claim 1, wherein said photo-crosslinking agent is riboflavin (vitamin B2).

7. (canceled)

8. The device according to claim 1, wherein said shapeable collagen gel mixture has a concentration of 3% and a solidifying pre-treatment time of 0.5˜5 minutes.
